(19) United States
(12) Patent Application Publication
(10) Pub. No.: US 2024/0100317 A1
(43) Pub. Date: Mar. 28, 2024

(54) CENTRAL LINE PROTECTOR APPARATUS

(71) Applicant: Mary Margaret Fedewa, DeWitt, MI (US)

(72) Inventor: Mary Margaret Fedewa, DeWitt, MI (US)

(21) Appl. No.: 18/472,075

(22) Filed: Sep. 21, 2023

Related U.S. Application Data
(60) Provisional application No. 63/409,009, filed on Sep. 22, 2022.

(51) Int. Cl. A61M 39/16; A61M 25/04

(52) U.S. Cl. CPC A61M 39/165; A61M 25/04; A61M 2240/00

(57) ABSTRACT

A central line protector apparatus includes (a) a central venous catheter (CVC); (b) a site protector providing a viewing shield surrounded by a protective frame; (c) an inlet provided on the site protector configured to covering and receiving the catheter; and (d) a protective sleeve attached to the site protector at the inlet and extending a predetermined length away from the site protector and configured to reduce undesired tampering of the catheter. The protective sleeve is configured to provide a passage for the catheter into the site protector, and into an entry point of the patient.

### CROSS REFERENCE TO RELATED APPLICATION

This application claims priority to and the benefit of U.S. Provisional Patent Application No. 63/409,009 titled “CENTRAL LINE PROTECTOR APPARATUS,” and filed on Sep. 22, 2022, the disclosure of which is incorporated herein by reference in its entirety for all purposes.

### TECHNICAL FIELD

The present disclosure relates generally to central lines and intravenous catheters.

### DESCRIPTION OF RELATED ART

A central intravenous line, referred to interchangeably as a central venous catheter (CVC), are often used in patients receiving treatment requiring multiple blood draws or IV medication. The difference between a typical IV and a CVC is that a CVC is a thin, flexible tube that is threaded along a large vein or subcutaneously until it reaches a large vein above the heart, typically the superior vena cava. However, the site of the entry port is vulnerable to infection and often requires careful and routine cleaning. Central line associated bloodstream infections (CLABSI) are common hospital-acquired infections and have been associated with mortality. A patient diagnosed with CLABSI may experience discomfort at or near the line of insertion, which can lead to higher temperatures due to infection/sepsis, pain, or further complications. Patients and families may also experience psychosocial effects including stress, economic hardship, and other difficulties related to hospitalized family members or children.

Pediatric patients can develop CLABSI due to a child's natural curiosity that leads them to play and tamper with the central line. This can be exacerbated by a desire to participate in socially normative play, which may result in perspiration and introduction of microorganisms to the entry port.

Many hospitals and clinics have policies and procedures to combat CLABSI, however the diagnosis of central line infections in children is difficult, especially when compounded by a severe illness or aggressive treatment such as chemotherapy. Often, the focus on CLABSI prevention has been in the ICU setting, however, many central lines are placed in patients treated in hospital units outside the ICU.

Despite preventative measures, central line infections continue to be commonplace. Further, many of these infections affect children which causes treatment interruptions, complications, longer hospital stays and higher mortality. There remains a need for an apparatus that prevents or reduces the likelihood of these infections related to central lines.

### SUMMARY

In one aspect, a central line protector apparatus includes (a) a central venous catheter (CVC), (b) a site protector providing a viewing shield surrounded by a protective frame, (c) an inlet provided on the site protector configured to covering and receiving the catheter, and (d) a protective sleeve attached to the site protector at the inlet and extending a predetermined length away from the site protector and configured to reduce undesired tampering of the catheter, where the protective sleeve is configured to provide a passage for the catheter into the site protector, and into an entry point of the patient.

The central line protector apparatus may also include further includes an adhesive element connected to the site protector configured to secure the apparatus to a patient for the CVC.

The central line protector apparatus may also include where the viewing shield is surrounded circumferentially by the protective frame.

The central line protector apparatus may also include where the viewing shield is made from a semi-rigid material.

The central line protector apparatus may also include where the viewing shield is substantially flat to prevent air or any foreign substance from entering.

The central line protector apparatus may also include where the viewing shield defines a convex bubble or dome shape extending out and away from a patient.

The central line protector apparatus may also include where the viewing shield is configured to promote breathability at a catheterization site while in use and reduce or prevent condensation or perspiration accumulating at a catheterization site by preventing the viewing shield from being in constant contact with a patient's skin.

The central line protector apparatus may also include where the viewing shield is relatively transparent and made of a clear material to enable a clear view of a catheterization site while in use without removing the site protector.

The central line protector apparatus may also include the viewing shield is formed of a material that is breathable and configured to prevent undesired contamination to a catheterization site while in use.

The central line protector apparatus may also include where the inlet is configured to receive the CVC and define a path for any tubing to remain accessible.

The central may also include further includes an attachment tab configured to secure the protective sleeve to the site protector.

The central line protector apparatus may also include where the protective sleeve is configured to receive and cover any external tubes that extend outside a patient while the CVC is inserted into the patient, where the protective sleeve surrounds the external tubes and provides protection external to the patient and decreases or prevents ingress of debris or microbes.

The central line protector apparatus may also include where the protective sleeve defines an access hole at a distal end configured to allow for insertion of CVC through the protective sleeve and into the site protector before insertion into a patient and the access hole is covered by a closing mechanism.

The central line protector apparatus may also include where the protective sleeve further includes an elastic band interposed between an attachment tab and an access hole, where the elastic band is configured to wrap around a circumference of the protective sleeve to cinch it closed and where the elastic band is placed near a distal end of the protective sleeve during delivery of medicine or blood draws and external ports on a distal end of one or more external tubes are accessible by exposing the ports through the access hole and the cinching effect of the elastic band is configured to hold the protective sleeve in place and prevent it from slipping beyond the exterior ports. Other technical features may be readily apparent to one skilled in the art from the following figures, descriptions, and claims.

In one aspect, the inlet is configured to cover and protect the CVC as it extends towards an insertion point.

In one aspect, the protective sleeve is made from silver impregnated fibers.

In one aspect, the protective sleeve is removable and interchangeable to allow for replacement with a clean and/or unused sleeve.

The central line protector apparatus may also include where the adhesive element is integrated with site protector.

The central line protector apparatus may also include a protective sleeve that is made from a breathable bacteriostatic cloth.

The central line protector apparatus may also include a protective sleeve that is pleated or accordion shaped configured to expand and contract in length to accommodate varying lengths of external tubes. Other technical features may be readily apparent to one skilled in the art from the following figures, descriptions, and claims.

For purposes of summarizing the disclosure, certain aspects, advantages, and novel features of the disclosure have been described herein. It is to be understood that not necessarily all such advantages may be achieved in accordance with any one particular embodiment of the disclosure. Thus, the disclosure may be embodied or carried out in a manner that achieves or optimizes one advantage or group of advantages as taught herein without necessarily achieving other advantages as may be taught or suggested herein. The features of the disclosure which are believed to be novel are particularly pointed out and distinctly claimed in the concluding portion of the specification. These and other features, aspects, and advantages of the present disclosure will become better understood with reference to the following drawings and detailed description.

### BRIEF DESCRIPTION OF THE DRAWINGS

The figures which accompany the written portion of this specification illustrate embodiments and method(s) of use for the present disclosure constructed and operative according to the teachings of the present disclosure.

FIG. 1 illustrates an example of a central line protector according to the present disclosure.

FIG. 2 illustrates the central line protector of FIG. 1 with a location of a catheterization site.

FIG. 3 illustrates a schematic side view of a central line protector having a dome or bubble protector according to the present disclosure.

FIG. 4 illustrates a schematic of an exemplary placement of a central line protector of the present disclosure positioned on a depiction of a patient.

### DETAILED DESCRIPTION

The following description is merely exemplary in nature and is not intended to limit the present disclosure, application, or uses. It should be understood through the drawings, corresponding reference numerals like or corresponding parts or features.

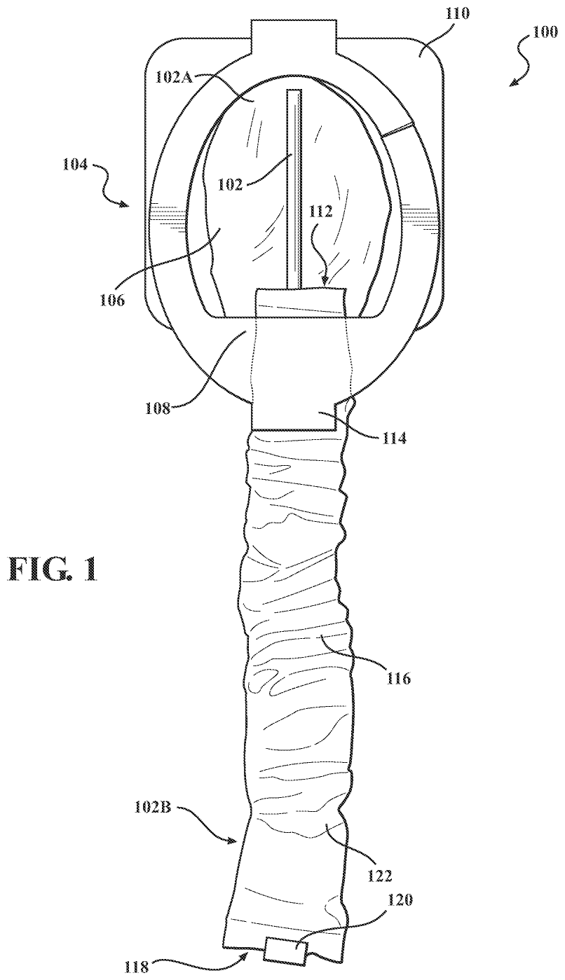

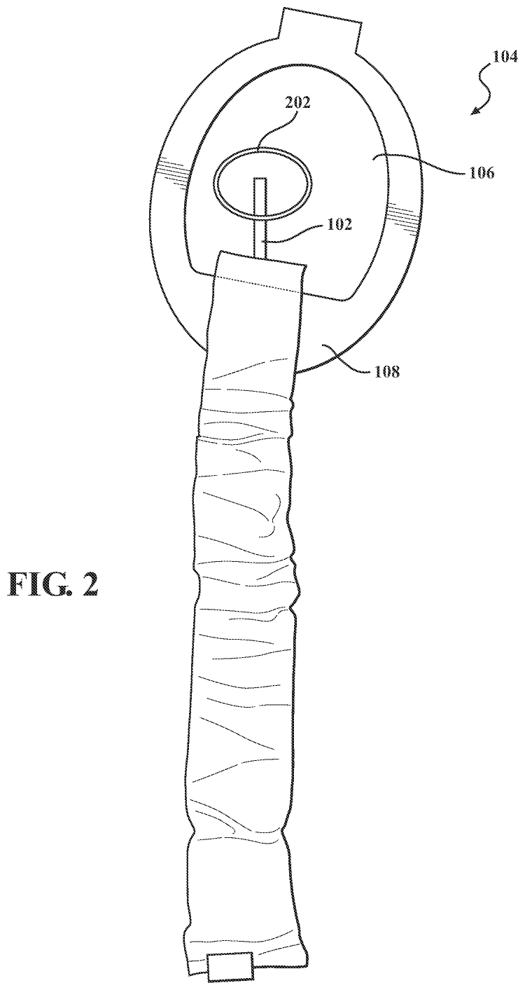

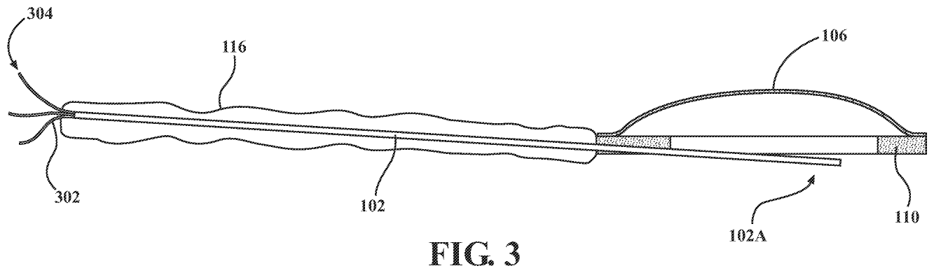

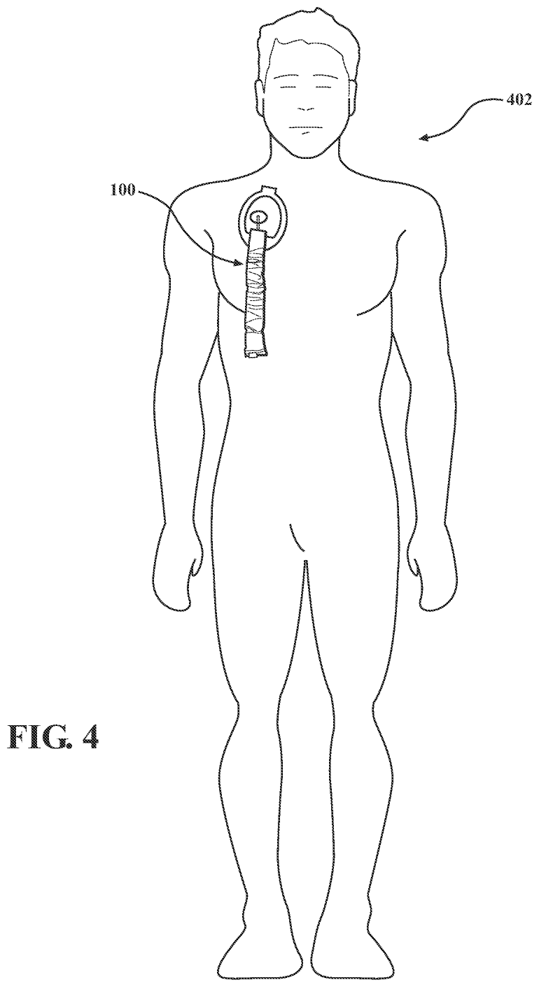

Referring to FIGS. 1-4, the present disclosure provides for a central line protector (CLP) apparatus 100. CLP apparatus 100 can be used with centrally placed intravenous lines referred to as a central venous catheter (CVC) 102, which typically is exposed from outside a patient 402 and is positioned through a vein extending to a larger vein near the heart. In an example, CLP apparatus 100 is applied at or near a site of insertion of CVC 102 which extends from a distal end 102B to a proximal end 102A. A distal end 102B of CVC 102 may include ports or access, which allow for the introduction of medication, fluids, or blood draws from a patient. CLP apparatus 100 can be configured to offer a sanitary yet breathable environment at the entry site of the CVC 102.

CLP apparatus 100 includes a site protector 104, which includes a viewing shield 106 surrounded circumferentially by a frame 108 and an optional adhesive element 110. Adhesive element 110 can be integrated with CLP apparatus 100 or added at the time of use so long as a something for holding CLP apparatus 100 in place is provided.

Viewing shield 106 may be made of a semi-rigid material, such as silicone or the like. It can be substantially flat to prevent air or any foreign substance from entering or formed into a convex bubble or dome extending out and away from the surface of a patient. Viewing shield 106 can be configured to reduce or prevent condensation or perspiration accumulating at a catheterization site 202 by preventing the shield from being in constant contact with the skin of a patient, thus promoting breathability at the catheterization site 202. Viewing shield 106 can be relatively transparent and may be made of a clear material to enable a clear view of the catheterization site 202 without removing site protector 104. In a further example, the material forming shield 106 is breathable and configured to prevent undesired contamination (e.g., microbial contaminants, viruses, bacteria, etc.) to the catheterization site 202.

Frame 108 provides structural support to site protector 104 while also framing shield 106 to an adhesive element 110. Frame 108 can be made from any material that is safe and relatively rigid to support the remaining components, yet flexible to be suitable for movement when in use.

Adhesive element 110 may be a film or any suitable adhesive configured to securing the apparatus to a desired location like a user's skin. Adhesive element 110 can be configured to be breathable yet strong enough to secure apparatus 100 to the surface of a patient and prevent undesired release or movement.

Site protector 104 further includes an inlet 112 configured to receive CVC 102 and defines a path for any associated tubing to remain accessible. The inlet 112 can be configured to cover and protect CVC 102 as it extends towards the insertion point. On an outer side of inlet 112, an attachment tab 114 is provided to secure a sleeve 116 to site protector 104. Attachment tab 114 provides a secure attachment site in which a sleeve 116 is adhered thereto which includes but is not limited to being sewn, glued, taped, crimped, or ultrasonically welded without compromising site protector 104.

In an example, a protective sleeve 116 is provided and configured to receive CVC 102 which is inserted into a patient during use. A portion of CVC 102 rests internal to a patient with larger and more robust external tubes 302 extending outside the body of a patient. Protective sleeve 116 surrounds external tubes 302 and provides protection for portions of the tubes that remain outside the body of a patient. This is achieved by covering external tubing 302 and decreasing ingress of debris or microbes. Protective sleeve 116 may be made of a breathable bacteriostatic cloth, for example, silver impregnated fibers. This is an added layer of protection by preventing undesired contamination. In a further example, protective sleeve 116 is removable and interchangeable to allow for replacement with a clean and/or unused sleeve. In an example, protective sleeve 116 may be pleated, or accordion shaped to expand and contract in length to accommodate varying lengths of external tubes 302.

Pediatric patients often touch, play, or engage with the external tubes 302 of CVC 102, which is an anticipated hazard of a condition that requires a CVC. However, tampering with the external tubes 302 is dangerous as it provides an opportunity for introduction of microbes and foreign objects/infections. Additionally, if external tubes 302 are manipulated too much, CVC 102 may become detached or loosened or added damage results to the insertion point or the vein itself. Due to the location of CVC 102 in the large vein near the heart, undesired tampering or damage can quickly lead to a large volume of blood rapidly escaping the patient resulting in shock or even the risk of “bleeding out” if not quickly treated. Protective sleeve 116 adds a structural support to protect external tubes 302 from tampering, while also concealing tubes 302, thus decreasing any urges to “play” with external tubes 302 or unintended dislodging or catching on external objects.

In an example, an access hole 118 is provided at a distal end of sleeve 116 to allow for insertion of CVC 102 through protective sleeve 116 and into site protector 104 before ultimately into the patient. In an example, a closing mechanism 120 is provided at access hole 118. The closing mechanism 120 may be a zipper, clip, re-closable slide seal, sliding zipper seal or combinations thereof among others.

In an example, an elastic band 122 is provided in sleeve 116 interposed between attachment tab 114 and access hole 118. Elastic band 122 can be configured to wrap around the circumference of protective sleeve 116 and cinches sleeve 116 closed. In an example, elastic band 122 is placed near the distal end of protective sleeve 116. During delivery of medicine or blood draws, external ports 304 on the distal end of external tubes 302 may be accessed by exposing the ports through access hole 118. The cinching effect of elastic band 122 holds protective sleeve 116 in place and prevents sleeve 116 from slipping beyond the exterior ports 304.

In an example elastic band 122 also provides for an additional level of security to prevent tampering with the external ports. Elastic band 122 may be slid beyond the distal ends of the ports such that if closing mechanism 120 is opened, the ports are still concealed within the cinched protective sleeve 116 behind elastic band 122.

It should be noted that the steps described in the method of use can be carried out in many different orders according to user preference. The use of “step of” should not be interpreted as “step for”, in the claims herein and is not intended to invoke the provisions of 35 U.S.C. § 112 (f). Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

What is claimed is:

1. A central line protector apparatus comprising:
(a) a central venous catheter (CVC);
(b) a site protector providing a viewing shield surrounded by a protective frame;
(c) an inlet provided on the site protector configured to covering and receiving the catheter; and
(d) a protective sleeve attached to the site protector at the inlet and extending a predetermined length away from the site protector and configured to reduce undesired tampering of the CVC;
wherein the protective sleeve is configured to provide a passage for the catheter into the site protector, and into an entry point of the patient.

2. The central line protector apparatus of claim 1, further comprising an adhesive element connected to the site protector configured to secure the apparatus to a patient for the CVC.

3. The central line protector apparatus of claim 2, wherein the adhesive element is integrated with site protector.

4. The central line protector apparatus of claim 1, wherein the viewing shield is surrounded circumferentially by the protective frame.

5. The central line protector apparatus of claim 1, wherein the viewing shield is made from a semi-rigid material.

6. The central line protector apparatus of claim 1, wherein the viewing shield is substantially flat to prevent air or any foreign substance from entering.

7. The central line protector apparatus of claim 1, wherein the viewing shield defines a convex bubble or dome shape extending out and away from a patient while preventing air or any foreign substance from entering while in use.

8. The central line protector apparatus of claim 1, wherein the viewing shield is configured to promote breathability at a catheterization site while in use and reduce or prevent condensation or perspiration accumulating at a catheterization site by preventing the viewing shield from being in constant contact with a patient's skin.

9. The central line protector apparatus of claim 1, wherein the viewing shield is relatively transparent and made of a clear material to enable a clear view of a catheterization site while in use without removing the site protector.

10. The central line protector apparatus of claim 1, the viewing shield is formed of a material that is breathable and configured to prevent undesired contamination to a catheterization site while in use.

11. The central line protector apparatus of claim 1, wherein the inlet is configured to receive the CVC and define a path for any tubing to remain accessible.

12. The central line protector apparatus of claim 11, wherein the inlet is configured to cover and protect the CVC as it extends towards an insertion point.

13. The central line protector apparatus of claim 1, further comprising an attachment tab configured to secure the protective sleeve to the site protector.

14. The central line protector apparatus of claim 1, wherein the protective sleeve is configured to receive and cover any external tubes that extend outside a patient while the CVC is inserted into the patient, wherein the protective sleeve surrounds the external tubes and provides protection external to the patient and decreases or prevents ingress of debris or microbes.

15. The central line protector apparatus of claim 14, wherein the protective sleeve is made from a breathable bacteriostatic cloth.

16. The central line protector apparatus of claim 15, wherein the protective sleeve is made from silver impregnated fibers.

17. The central line protector apparatus of claim 15, wherein the protective sleeve is removable and interchangeable to allow for replacement with a clean and/or unused sleeve.

18. The central line protector apparatus of claim 15, wherein the protective sleeve is pleated or accordion shaped configured to expand and contract in length to accommodate varying lengths of external tubes.

19. The central line protector apparatus of claim 1, wherein the protective sleeve defines an access hole at a distal end configured to allow for insertion of CVC through the protective sleeve and into the site protector before insertion into a patient and the access hole is covered by a closing mechanism.

20. The central line protector apparatus of claim 1, wherein the protective sleeve further includes an elastic band interposed between an attachment tab and an access hole, wherein the elastic band is configured to wrap around a circumference of the protective sleeve to cinch it closed and wherein the elastic band is placed near a distal end of the protective sleeve during delivery of medicine or blood draws and external ports on a distal end of one or more external tubes are accessible by exposing the ports through the access hole and the cinching effect of the elastic band is configured to hold the protective sleeve in place and prevent it from slipping beyond the exterior ports.
